(19) United States
(12) Patent Application Publication
(10) Pub. No.: US 2023/0398140 A1
(43) Pub. Date: Dec. 14, 2023

(54) OCULAR-TENSION-INCREASING AGENT CAPABLE OF BEING DEPRIVED OF DRUG ACTIVITY BY TEMPERATURE CONTROL

(71) Applicants: UNIVERSITY OF YAMANASHI, Yamanashi (JP); TOKYO UNIVERSITY OF SCIENCE FOUNDATION, Tokyo (JP)

(72) Inventors: Yuji TANAKA, Yamanashi (JP); Kenji KASHIWAGI, Yamanashi (JP); Shigehito OSAWA, Tokyo (JP)

(73) Assignee: UNIVERSITY OF YAMANASHI, Yamanashi (JP)

(21) Appl. No.: 18/034,254

(22) PCT Filed: Oct. 28, 2021
(86) PCT No.: PCT/JP2021/039837

§ 371 Date: Jul. 21, 2023

(30) Foreign Application Priority Data
Oct. 29, 2020 (JP) 2020-181848

(51) Int. Cl. A61K 31/785; A61K 9/00; A61K 31/765; A61P 27/02

(52) U.S. Cl. CPC A61K 31/785; A61K 9/0048; A61K 31/765; A61P 27/02

(57) ABSTRACT

The disclosure provides an ocular tension increasing agent containing a temperature responsive polymer. Ocular tension can be increased by administering an ocular tension increasing agent according to the present invention to a predetermined region of an eye and ocular tension can be controlled, if necessary, by temperature.

### TECHNICAL FIELD

The present invention relates to an ocular tension increasing agent, and particularly to an ocular tension increasing agent whose medicinal effect can be cancelled by temperature control.

### BACKGROUND ART

For an eyeball to maintain a normal shape and function, it is considered necessary that ocular tension is kept at a constant value (for example, approximately 10 to 20 mmHg). In contrast, a treatment for decreasing ocular tension is actively applied to glaucoma, which has a high risk of blindness and is developed or proceeds if ocular tension increases. However, if ocular tension decreases, an eyeball deforms, with the result that not only a cosmetic problem but also a serious vision disorder such as maculopathy and detachment of ciliary body, may occur (FIG. 1). Ocular hypotension is a cause of a serious visual defect but there is no standard treatment for the hypotension at present.

Ocular tension is controlled by “production of aqueous humor” in ciliary body epithelial cells and “drainage of aqueous humor” from the angle present in the border region between the cornea and the iris via Schlemm's canal. Ocular hypotension may be caused by a decrease in aqueous humor production due to disorder of unpigmented epithelial cells of the ciliary body and accelerated aqueous humor drainage from the angle. To deal with a decrease of aqueous humor production, it is necessary to reproduce cells and tissues. Since the reproduction of cells and tissues are extremely difficult, decreasing drainage of aqueous humor from the angle is a means actually taken.

At present, there are no ocular tension increasing agents approved as pharmaceuticals. Examples of a surgical treatment include (1) intraocular injection of a viscoelastic substance such as hyaluronic acid or air, (2) suture of the angle and (3) adhesion of the angle by laser irradiation.

Non Patent Literature 1 discloses a method of injecting resin beads into aqueous humor and charging the angle and Schlemm's canal with the beads, at the level of animal experiments.

### CITATION LIST

### Non Patent Literatures

Non Patent Literature 1: Investigative Ophthalmology & Visual Science, January 2013, Vol. 54, No. 1, P762-, “Elevated Intraocular Pressure Causes Inner Retinal Dysfunction Before Cell Loss in a Mouse Model of Experimental Glaucoma”, Benjamin J. Frankfort etc.

### SUMMARY OF INVENTION

Non Patent Literature 1 discloses a method of physically blocking the outlet of the aqueous humor with resin beads. This is a suitable approach for increasing ocular tension. However, resin beads cannot be removed from the outlet of aqueous humor. If the ocular tension excessively increases, conceivably ocular tension cannot be decreased. Accordingly, a conventional treatment requires a technique avoiding an excessive increase of ocular tension.

The present inventors found a method for increasing ocular tension by administering an ocular tension increasing agent containing a temperature responsive polymer to the aqueous humor; a method that can decrease ocular tension, if excessively increased, by cooling an eyeball; and a composition containing a temperature responsive polymer that is effectively used for these methods.

Ocular tension can be increased by administering the ocular tension increasing agent according to the present invention to a predetermined region of an eyeball and can be controlled by temperature.

A medicinal effect produced by the ocular tension increasing agent may be cancelled by controlling temperature. The temperature responsive polymer aggregates at the temperature of intraocular circulating fluid present in the ocular tissue of a subject and may be linearized (more specifically, solubilized) as the temperature of the intraocular circulating fluid is decreased. The ocular tissue of the subject may have a first region to which the temperature responsive polymer is to be administered and a second region fluidly communicated with the first region. The intraocular circulating fluid may flow from the first region to the second region. The flow of the intraocular circulating fluid from the first region to the second region may be blocked by the temperature responsive polymer aggregated. The blockage of the flow of the intraocular circulating fluid may be removed with linearization of the temperature responsive polymer aggregated. The ocular tension increasing agent may contain the temperature responsive polymer in an amount that blocks the flow of the intraocular circulating fluid from the first region to the second region. The first region may be the angle. The second region may be Schlemm's canal. The temperature responsive polymer may be an oxazoline polymer.

An object of the present invention is to provide a pharmaceutical composition containing an ocular tension increasing agent as mentioned above for treating ocular hypotension.

The pharmaceutical composition according to the present invention can treat ocular hypotension.

Another object of the present invention is to provide a method for treating ocular hypotension having a step of administering an ocular tension increasing agent or pharmaceutical composition as mentioned above to the ocular tissue of a subject that needs treatment for ocular hypotension.

Ocular hypotension can be treated by the method for treating ocular hypotension according to the present invention.

According to the present invention, for example, the following inventions are provided.

(1) An ocular tension increasing agent containing a temperature responsive polymer.

(2) The ocular tension increasing agent according to (1), whose medicinal effect can be cancelled by temperature control.

(3) The ocular tension increasing agent according to (1) or (2), wherein the temperature responsive polymer aggregates at the temperature of intraocular circulating fluid present in the ocular tissue of a subject and is linearized by decreasing the temperature of the intraocular circulating fluid.

(4) The ocular tension increasing agent according to (3), wherein the ocular tissue of the subject has a first region in which the temperature responsive polymer is to be administered and a second region fluidly communicated with the first region,
the intraocular circulating fluid flows from the first region to the second region,
the flow of the intraocular circulating fluid from the first region to the second region is blocked by the temperature responsive polymer aggregated, and
the blockage of the flow of the intraocular circulating fluid is removed with linearization of the temperature responsive polymer aggregated.

(5) The ocular tension increasing agent according to (4), containing a temperature responsive polymer in an amount that blocks flow of the intraocular circulating fluid from the first region to the second region.

(6) The ocular tension increasing agent according to (4) or (5), wherein
the first region is the angle, and
the second region is Schlemm's canal.

(7) The ocular tension increasing agent according to any one of (1) to (6), wherein the temperature responsive polymer is an oxazoline polymer.

(8) A pharmaceutical composition containing the ocular tension increasing agent according to any one of (1) to (7), for treating ocular hypotension.

(11) A composition for use in a method for increasing ocular tension in a subject, containing a temperature responsive polymer.

(12) The composition according to (11), wherein the temperature responsive polymer has the lower critical solution temperature (LCST) between 0° C. and 37° C.

(13) The composition according to (11) or (12), wherein the temperature responsive polymer has the lower critical solution temperature (LCST) between 15° C. and 30° C.

(14) The composition according to any one of (11) to (13), wherein the method for increasing ocular tension in a subject includes administering the temperature responsive polymer to the anterior chamber of an eyeball of the subject and thereafter cooling (for example, icing) the eyeball.

(15) The composition according to any one of (11) to (14), further containing a copolymer of an uncharged hydrophilic polymer and a temperature responsive polymer.

(16) The composition according to any one of (11) to (15), wherein the temperature responsive polymer is polyoxazoline.

(17) The composition according to any one of (11) to (16), wherein the uncharged hydrophilic polymer is polyalkylene glycol.

(18) A composition containing polyoxazoline (POx) and a block copolymer (POxPEG) containing a polyethylene glycol block and a polyoxazoline block.

(19) The composition according to (18), wherein the weight ratio of Pox to POxPEG is from 90:10 to 99:1.

(20) The composition according to (18) or (19), for use in the method for increasing ocular tension in a subject.

(31) The composition according to any one of the above items, wherein the method for increasing ocular tension in a subject includes administering a temperature responsive polymer to an eyeball (particularly anterior chamber) of a subject.

(32) The composition according to any one of the above items, wherein the method for increasing ocular tension in a subject includes administering a temperature responsive polymer to an eyeball (particularly anterior chamber) of a subject and thereafter cooling the eyeball.

(33) The composition according to any one of the above items, wherein the method for increasing ocular tension in a subject includes administering a temperature responsive polymer to an eyeball (particularly anterior chamber) of a subject and thereafter subjecting the eyeball to at least one cycle of eyeball cooling and warming treatments.

(34) The composition according to any one of the above items, wherein the method for increasing ocular tension in a subject includes administering a temperature responsive polymer to an eyeball (particularly anterior chamber) of a subject and thereafter subjecting the eyeball to at least one cycle of eyeball cooling and warming treatments, thereby controlling the ocular tension of the treated eye of the subject to be 20 mmHg or less.

(35) The method according to any one of the above items, wherein the method for increasing ocular tension in a subject further includes warming the temperature responsive polymer before administration to a temperature that is suitable for administration to an eyeball and equal to or more than the LCST.

(41) The composition according to any one of the above items, wherein the temperature responsive polymer is a compound represented by the following formula (X):

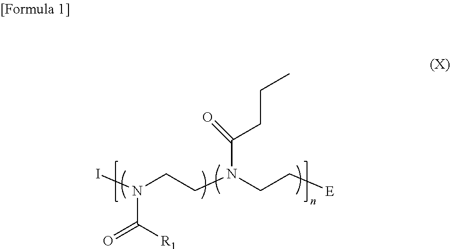

wherein R₁ represents a methyl group, an ethyl group, a propyl group (for example, a n-propyl group or an isopropyl group) or a cycloalkyl group; I represents hydrogen, alkyl, alkynyl, alkenyl, alkyloxy, a protecting group or a polymerizable group; E represents hydrogen, alkyl, alkynyl, alkenyl, alkyloxy, a protecting group or a polymerizable group; and n represents an integer of 30 to 300.

(42) The composition according to (41), wherein the compound is a block copolymer.

(43) The composition according to (41), wherein the compound is a statistical copolymer.

(44) The composition according to (41), wherein the compound is poly(2-oxazoline).

(51) A composition for use in the method for increasing ocular tension in a subject, containing a first temperature responsive polymer and a second temperature responsive polymer.

(52) The composition according to (51), wherein the second temperature responsive polymer is a block copolymer of an uncharged hydrophilic polymer and a temperature responsive polymer {the first temperature responsive polymer can be a homopolymer}.

(53) The composition according to (51) or (52), wherein the first temperature responsive polymer is a compound represented by the following formula (X).

(54) The composition according to any one of (51) to (53), wherein the first temperature responsive polymer is poly(2-oxazoline).

(55) The composition according to any one of (51) to (54), wherein the second temperature responsive polymer is a block copolymer of an uncharged hydrophilic polymer and a compound represented by the following formula (X).

(56) The composition according to any one of (51) to (55), wherein the second temperature responsive polymer is a block copolymer of a polyalkylene glycol and a compound represented by formula (X).

(57) The composition according to any one of (51) to (56), wherein the second temperature responsive polymer is a block copolymer of polyethylene glycol and a compound represented by formula (X).

(58) The composition according to any one of (51) to (57), wherein the second temperature responsive polymer is a block copolymer of polyethylene glycol and poly(2-oxazoline).

(59) The compound according to (53), wherein the second temperature responsive polymer is a block copolymer of an uncharged hydrophilic polymer and a compound represented by the following formula (X).

(60) The compound according to (54), wherein the second temperature responsive polymer is a block copolymer of an uncharged hydrophilic polymer and a compound represented by the following formula (X).

(61) The compound according to (53), wherein the second temperature responsive polymer is a block copolymer of a polyalkylene glycol and a compound represented by formula (X).

(62) The compound according to (54), wherein the second temperature responsive polymer is a block copolymer of a polyalkylene glycol and a compound represented by formula (X).

(63) The compound according to (53), wherein the second temperature responsive polymer is a block copolymer of polyethylene glycol and a compound represented by formula (X).

(64) The compound according to (54), wherein the second temperature responsive polymer is a block copolymer of polyethylene glycol and a compound represented by formula (X).

(65) The compound according to (53), wherein the second temperature responsive polymer is a block copolymer of polyethylene glycol and a compound represented by formula (X).

(66) The compound according to (54), wherein the second temperature responsive polymer is a block copolymer of polyethylene glycol and a compound represented by formula (X).

(67) The compound according to (53), wherein the second temperature responsive polymer is a block copolymer of polyethylene glycol and poly(2-oxazoline).

(68) The compound according to (54), wherein the second temperature responsive polymer is a block copolymer of polyethylene glycol and poly(2-oxazoline).

(81) The composition according to any one of (51) to (68), wherein the weight ratio of the first temperature responsive polymer to the second temperature responsive polymer is from 90:10 to 99:1.

(82) The composition according to any one of the above items, wherein the temperature responsive polymer forms particles having a particle size of 1 to 10 μm in an aqueous solution of 37° C. (for example, in a saline solution or in a formulation).

(83) The composition according to any one of (51) to (68), wherein the temperature responsive polymer forms particles having a particle size of 1 to 10 μm in an aqueous solution of 37° C. (for example, in a saline solution or in a formulation).

(84) The composition according to (81), wherein the temperature responsive polymer forms particles having a particle size of 1 to 10 μm in an aqueous solution of 37° C. (for example, in a saline solution or in a formulation).

(101) The composition according to any one of the above items, having a temperature within the range of the LCST to 37° C.

(102) The composition according to any one of the above items, having a temperature within the range of 0° C. to less than the LCST.

(111) A composition for use in treating a subject having low ocular tension or ocular hypotension, containing a temperature responsive polymer.

(112) The composition according to (111), wherein the temperature responsive polymer has the lower critical solution temperature (LCST) between 0° C. and 37° C.

(113) The composition according to (111) or (112), wherein the temperature responsive polymer has the lower critical solution temperature (1LCST) between 15° C. and 30° C.

(114) The composition according to any one of (111) to (113), wherein the method for increasing ocular tension in a subject includes administering the temperature responsive polymer to the anterior chamber of an eyeball of a subject and thereafter cooling the eyeball of the subject.

(115) The composition according to any one of (111) to (114), further containing a copolymer of an uncharged hydrophilic polymer and a temperature responsive polymer.

(116) The composition according to any one of (111) to (115), wherein the temperature responsive polymer is polyoxazoline.

(117) The composition according to any one of (111) to (116), wherein the uncharged hydrophilic polymer is a polyalkylene glycol.

(118) A composition containing polyoxazoline (POx) and a block copolymer (POxPEG) containing a polyethylene glycol block and a polyoxazoline block.

(119) The composition according to (118), wherein the weight ratio of POx to POxPEG is from 90:10 to 99:1.

(120) The composition according to (118) or (119), for use in the method for increasing ocular tension in a subject.

(131) The composition according to any one of the above items, wherein the method for increasing ocular tension in a subject includes administering a temperature responsive polymer to an eyeball (particularly anterior chamber) of a subject.

(132) The composition according to any one of the above items, wherein the method for increasing ocular tension in a subject includes administering a temperature responsive polymer to an eyeball (particularly anterior chamber) of a subject and thereafter cooling the eyeball.

(133) The composition according to any one of the above items, wherein the method for increasing ocular tension in a subject includes administering a temperature responsive polymer to an eyeball (particularly anterior chamber) of a subject and thereafter subjecting the eyeball to at least one cycle of eyeball cooling and warming treatments.

(134) The composition according to any one of the above items, wherein the method for increasing ocular tension in a subject includes administering a temperature responsive polymer to an eyeball (particularly anterior chamber) of a subject and thereafter subjecting the eyeball to at least one cycle of eyeball cooling and warming treatments, thereby controlling the ocular tension of the treated eye of the subject to be 20 mmHg or less.

(135) The method according to any one of the above items, wherein the method for increasing ocular tension in a subject further includes warming the temperature responsive polymer before administration to a temperature that is suitable for administration to an eyeball and the LCST or more.

(141) The composition according to any one of the above items, wherein the temperature responsive polymer is a compound represented by the following formula (X):

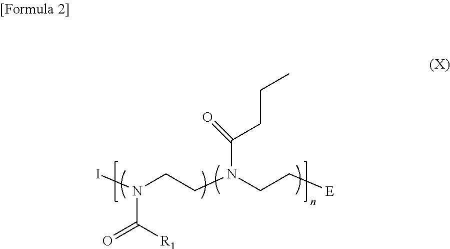

wherein R₁ represents a methyl group, an ethyl group, a propyl group (for example, a n-propyl group or an isopropyl group) or a cycloalkyl group; I represents hydrogen, alkyl, alkynyl, alkenyl, alkyloxy, a protecting group or a polymerizable group; E represents hydrogen, alkyl, alkynyl, alkenyl, alkyloxy, a protecting group or a polymerizable group; and n represents an integer of 3 to 300.

(142) The composition according to (141), wherein the compound is a block copolymer.

(143) The composition according to (141), wherein the compound is a statistical copolymer.

(144) The composition according to (141), wherein the compound is poly(2-oxazoline).

(151) A composition for use in a method for increasing ocular tension in a subject, containing a first temperature responsive polymer and a second temperature responsive polymer.

(152) The composition according to (151), wherein the second temperature responsive polymer is a block copolymer of an uncharged hydrophilic polymer and a temperature responsive polymer {the first temperature responsive polymer can be a homopolymer}.

(153) The composition according to (151) or (152), wherein the first temperature responsive polymer is a compound represented by formula (X).

(154) The composition according to any one of (151) to (153), wherein the first temperature responsive polymer is poly(2-oxazoline).

(155) The composition according to any one of (151) to (154), wherein the second temperature responsive polymer is a block copolymer of an uncharged hydrophilic polymer and a compound represented by formula (X).

(156) The composition according to any one of (151) to (155), wherein the second temperature responsive polymer is a block copolymer of a polyalkylene glycol and a compound represented by formula (X).

(157) The composition according to any one of (151) to (156), wherein the second temperature responsive polymer is a block copolymer of polyethylene glycol and a compound represented by formula (X).

(158) The composition according to any one of (151) to (157), wherein the second temperature responsive polymer is a block copolymer of polyethylene glycol and poly(2-oxazoline).

(159) The compound according to (153), wherein the second temperature responsive polymer is a block copolymer of an uncharged hydrophilic polymer and a compound represented by the formula (X).

(160) The compound according to (154), wherein the second temperature responsive polymer is a block copolymer of an uncharged hydrophilic polymer and a compound represented by the formula (X).

(161) The compound according to (153), wherein the second temperature responsive polymer is a block copolymer of a polyalkylene glycol and a compound represented by formula (X).

(162) The compound according to (154), wherein the second temperature responsive polymer is a block copolymer of a polyalkylene glycol and a compound represented by formula (X).

(163) The compound according to (153), wherein the second temperature responsive polymer is a block copolymer of polyethylene glycol and a compound represented by formula (X).

(164) The compound according to (154), wherein the second temperature responsive polymer is a block copolymer of polyethylene glycol and a compound represented by formula (X).

(165) The compound according to (153), wherein the second temperature responsive polymer is a block copolymer of polyethylene glycol and a compound represented by formula (X).

(166) The compound according to (154), wherein the second temperature responsive polymer is a block copolymer of polyethylene glycol and a compound represented by formula (X).

(167) The compound according to (153), wherein the second temperature responsive polymer is a block copolymer of polyethylene glycol and poly(2-oxazoline).

(168) The compound according to (154), wherein the second temperature responsive polymer is a block copolymer of polyethylene glycol and poly(2-oxazoline).

(181) The composition according to any one of (151) to (168), wherein the weight ratio of the first temperature responsive polymer to the second temperature responsive polymer is from 90:10 to 99:1.

(182) The composition according to any one of (151) to (168), wherein the weight ratio of the first temperature responsive polymer to the second temperature responsive polymer is from 95:10 to 99:1.

(183) The composition according to any one of (151) to (168), wherein the weight ratio of the first temperature responsive polymer to the second temperature responsive polymer is from 97:10 to 99:1.

(184) The composition according to any one of (151) to (168), wherein the weight ratio of the first temperature responsive polymer to the second temperature responsive polymer is p:100−p, where p is about 98.

(191) The composition according to any one of the above items, wherein the temperature responsive polymer forms particles having a particle size of 1 to 10 μm in an aqueous solution of 37° C. (for example, in a saline solution or in a formulation).

(192) The composition according to any one of (151) to (168), wherein the temperature responsive polymer forms particles having a particle size of 1 to 10 μm in an aqueous solution of 37° C. (for example, in a saline solution or in a formulation).

(193) The composition according to any one of (181) to (184), wherein the temperature responsive polymer forms particles having a particle size of 1 to 10 μm in an aqueous solution of 37° C. (for example, in a saline solution or in a formulation).

(194) The composition according to (182), wherein the temperature responsive polymer forms particles having a particle size of 1 to 10 μm in an aqueous solution of 37° C. (for example, in a saline solution or in a formulation).

(195) The composition according to (183), wherein the temperature responsive polymer forms particles having a particle size of 1 to 10 μm in an aqueous solution of 37° C. (for example, in a saline solution or in a formulation).

(196) The composition according to (184), wherein the temperature responsive polymer forms particles having a particle size of 1 to 10 μm in an aqueous solution of 37° C. (for example, in a saline solution or in a formulation).

(201) The composition according to any one of the above items, having a temperature within the range of the LCST to 37° C.

(202) The composition according to any one of the above items, having a temperature within the range of 0° C. to less than the LCST.

(211) The composition according to any one of the above items, wherein a subject having low ocular tension or ocular hypotension further has a complication.

(212) The composition according to (211), wherein the complication is at least one selected from the group consisting of detachment of ciliary body, a retinal disorder such as retinal detachment and macular degeneration, and iridocyclitis.

(301) A method for increasing ocular tension in a subject, including administering a temperature responsive polymer or an aqueous formulation containing a temperature responsive polymer to the patient's eyeball (particularly aqueous humor).

(302) The composition according to (301), wherein the temperature responsive polymer has the lower critical solution temperature (LCST) between 0° C. and 37° C.

(303) The composition according to (301) or (302), wherein the temperature responsive polymer has the lower critical solution temperature (LCST) between 15° C. and 30° C.

(304) The composition according to any one of (301) to (303), wherein the method for increasing ocular tension in a subject includes administering the temperature responsive polymer to the anterior chamber of an eyeball of a subject and thereafter cooling (for example, icing) the eyeball of the subject.

(305) The composition according to any one of (301) to (304), further containing a copolymer of an uncharged hydrophilic polymer and a temperature responsive polymer.

(306) The composition according to any one of (301) to (305), wherein the temperature responsive polymer is polyoxazoline.

(307) The composition according to any one of (301) to (306), wherein the uncharged hydrophilic polymer is a polyalkylene glycol.

(308) A composition containing polyoxazoline (POx) and a block copolymer (POxPEG) containing a polyethylene glycol block and a polyoxazoline block.

(309) The composition according to (308), wherein the weight ratio of POx to POxPEG is from 90:10 to 99:1.

(310) The composition according to (308) or (309) for use in the method for increasing ocular tension in a subject.

(321) The composition according to any one of the above items, wherein the method for increasing ocular tension in a subject includes administering a temperature responsive polymer to an eyeball (particularly anterior chamber) of the subject.

(322) The composition according to any one of the above items, wherein the method for increasing ocular tension in a subject includes administering a temperature responsive polymer to an eyeball (particularly anterior chamber) of the subject and thereafter cooling the eyeball.

(323) The composition according to any one of the above items, wherein the method for increasing ocular tension in a subject includes administering a temperature responsive polymer to an eyeball (particularly anterior chamber) of the subject and thereafter subjecting the eyeball to at least one cycle of eyeball cooling and warming treatments.

(324) The composition according to any one of the above items, wherein the method for increasing ocular tension in a subject includes administering a temperature responsive polymer to an eyeball (particularly anterior chamber) of the subject and thereafter subjecting the eyeball to at least one cycle of eyeball cooling and warming treatments, thereby controlling the ocular tension of the treated eye of the subject to be 20 mmHg or less.

(325) The method according to any one of the above items, wherein the method for increasing ocular tension in a subject further includes warming the temperature responsive polymer before administration to a temperature that is suitable for administration to an eyeball and the LCST or more.

(331) The composition according to any one of the above items, wherein the temperature responsive polymer is a compound represented by the following formula (X):

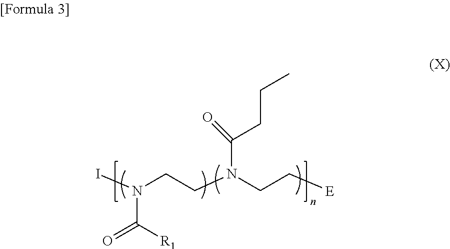

wherein R₁ represents a methyl group, an ethyl group, a propyl group (for example, a n-propyl group or an isopropyl group) or a cycloalkyl group; I represents hydrogen, alkyl, alkynyl, alkenyl, alkyloxy, a protecting group or a polymerizable group; E represents hydrogen, alkyl, alkynyl, alkenyl, alkyloxy, a protecting group or a polymerizable group; and n represents an integer of 3 to 300.

(332) The composition according to (331), wherein the compound is a block copolymer.

(333) The composition according to (331), wherein the compound is a statistical copolymer.

(334) The composition according to (331), wherein the compound is poly(2-oxazoline).

### BRIEF DESCRIPTION OF DRAWINGS

FIG. 1 is a drawing explaining effects of ocular hypotension.

FIG. 2 schematically showing the effect of the ocular tension increasing agent of the present invention.

FIG. 3 is a schematic diagram of a temperature responsive polymer 10 administered within an ocular tissue 1. FIG. 3B is a schematic diagram illustrating that an aggregated temperature responsive polymer 10A affects the flow of intraocular circulating fluid in the ocular tissue 1 of a subject. FIG. 3C shows a linearized temperature responsive polymer 10B.

FIG. 4 is a graph showing ocular tension of rats to which a temperature responsive polymer is intraocularly injected.

FIG. 5 shows microscope images of various mixed solutions containing a temperature responsive polymer and placed on slide glass surfaces.

FIG. 6 shows disappearance of particles in various mixed solutions containing a temperature responsive polymer by icing and reformation of particles by warming.

FIG. 7 shows the results of increases of ocular tension after various mixed solutions containing a temperature responsive polymer were administrated to the anterior chamber. Plots are increased ocular tensions (ΔIOP) of eyeballs administered with mixed solutions containing Pox and POxPEG in the indicated ratios. Gray boxes are box plots.

FIG. 8 shows the presence or absence of cloudiness in eyeballs after various mixed solutions containing a temperature responsive polymer were administered to the anterior chambers.

FIG. 9 shows increases of ocular tension after mixed solutions containing a temperature responsive polymer were administered to the anterior chamber and cancellation of increases of ocular tension by icing. The plots are increased ocular tension values (ΔIOP) in individual eyeballs injected with a mixed solution containing Pox and POxPEG in a molar ratio of 98:2. Gray boxes are box plots.

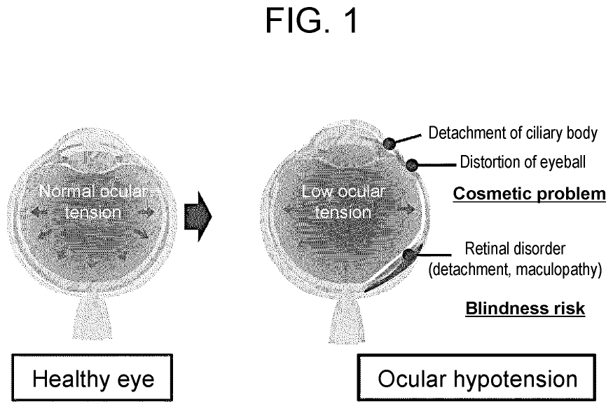

### DESCRIPTION OF EMBODIMENTS

### Definition

For the sake of convenience, the terms specifically used in the present application are collectively defined herein. Unless otherwise specified, all technical terms and scientific terms to be used in the present application have the same meanings as understood by those skilled in the art to which the present invention pertains. Unless otherwise specified in the specification, the singular forms “a” “an” and “the” include plural references.

Numeric ranges and parameters shown in the present invention are approximate values, but numerical values shown in specific Examples are disclosed as accurately as possible. However, any one of the numerical values basically contains a specific error inevitably derived from a standard deviation of each of values measured in experiments. The term “about” used in the specification generally refers to the range within 10%, 5%, 1% or 0.5% of a given value or range. Alternatively, the term “about” understood by those skilled in the art is a value within an acceptable standard error.

The term “test subject”, “subject” and “patient” are interchangeably used in the specification and refer to an animal including a human that can be treated by a synthetic peptide and/or method of the present invention. Both genders (male and females) are intended by the term “test subject”, “subject” or “patient”, unless otherwise specified. Thus, the term “test subject”, “subject” or “patient” inclusively refers to any mammal that can obtain a benefit from a therapeutic method disclosed by the invention. Examples of the “test subject”, “subject” or “patient” include, but are not limited to, a human, a rat, a mouse, a guinea pig, a monkey, a pig, a goat, a cow, a horse, a dog, a cat, a bird and a chicken. In a preferred exemplary embodiment, the “test subject”, “subject” or “patient” is a human.

In the specification, “ocular hypotension” refers to a decrease of ocular tension. The normal range of ocular tension is defined to be approximately 10 to 20 mmHg. A low ocular tension refers to a tension of less than 10 mmHg and thus ocular hypotension is defined as an intraocular pressure (IOP) of less than 6.5 mmHg. According to clinical definition, ocular hypotension is low IOP sufficient to lead to a pathologic condition (for example, blindness). Blindness derived from low IOP may be caused by corneal edema, astigmatism, cystoid macular edema, maculopathy or other disease states. Low pressure maculopathy is characterized by low IOP associated with abnormalities in ocular fundus including choroidal fold, acute edema of the optic disk and tortuosity of the vessel.

In the specification, the term “treatment” refers to a treatment that can bring a medical benefit to a subject having a disease or an unfavorable physical condition. The medical benefit may be brought by reducing a speed of worsening a disease or an unfavorable physical condition, preventing worsening a disease or an unfavorable physical condition, improving a physical condition from a disease or an unfavorable condition and(or) curing or inducing a remission of a disease or an unfavorable physical condition. Accordingly, a treatment for low ocular tension or ocular hypotension refers to reducing a speed of worsening low ocular tension or ocular hypotension, preventing worsening low ocular tension or ocular hypotension, improving a physical condition from low ocular tension or ocular hypotension, or curing or inducing a remission of low ocular tension or ocular hypotension. Also, the treatment for low ocular tension or ocular hypotension may further includes reducing a risk of a complication caused by low ocular tension or ocular hypotension and preventing onset of a complication. Examples of the complication include corneal edema, astigmatism, cystoid macular edema, maculopathy, other disease states caused by ocular hypotension and blindness. The complications refer to other diseases or disease states caused by low ocular tension or ocular hypotension.

In the specification, the term “temperature responsive polymer” refers to a polymer soluble in water at a low temperature but becomes hydrophobic at a predetermined temperature (lower critical solution temperature (LCST)) or more to produce a white turbidity or precipitates.

Examples of the ocular hypotension include detachment of ciliary body, eyeball distortion and retinal disorder (peeling, macular degeneration). Cosmetic problems and risk of blindness may be caused by ocular hypotension.

Now, embodiments of the present invention will be described. The following embodiments are just examples, and thus, the range of the present invention is not limited to those disclosed in the following embodiments. Note that, for the sake of brevity, explanation of the similar content will be appropriately omitted.

Ocular Tension Increasing Agent

In the specification, the ocular tension increasing agent refers to a composition for use in increasing ocular tension in a subject. The ocular tension increasing agent according to the embodiment contains a temperature responsive polymer. The ocular tension increasing agent disclosed herein can increase ocular tension by injecting the agent into an eyeball, particularly the anterior chamber thereof. The medicinal effect of the ocular tension increasing agent can be cancelled by controlling the temperature of a temperature responsive polymer (particularly through cooling), that is, an ocular tension increasing effect can be cancelled. More specifically, the ocular tension increasing agent can increase ocular tension by injecting it in the anterior chamber and an increase of ocular tension can be decreased or cancelled by cooling the eyeball having an increased ocular tension. For this purpose, a temperature responsive polymer, which has the LCST within the temperature range from 0° C. to body temperature (for example, 36° C. to 37° C.), may be preferably used. The temperature responsive polymer having the LCST within the temperature range become insoluble at the body temperature to block an aqueous-humor outlet, thereby blocking drainage of the aqueous humor from the eyeball. If the eyeball is cooled to less than the LCST within a physiological acceptable range, the temperature responsive polymer is dissolved in aqueous humor, with the result that blockage of drainage of aqueous humor from the eyeball is terminated. As described above, according to the disclosure of the invention, the ocular tension of a subject is increased by injecting a composition containing a temperature responsive polymer having the LCST within the temperature range of 0° C. to body temperature (for example, 36° C. to 37° C.) or ocular tension increased can be canceled by cooling the eyeball.

The aqueous formulation to be administered may have a temperature from the LCST to body temperature (for example, 36 to 38° C.). The aqueous formulation is an aqueous solution having a temperature responsive polymer dissolved therein at a temperature of less than the LCST. An aqueous solution having a temperature responsive polymer dissolved therein can be intraocularly administered. When the temperature responsive polymer administered is warmed up to the temperature of the eyeball, particles of the temperature responsive polymer can be formed, with the result that the particles block drainage of aqueous humor from an aqueous humor outlet and can induce an increase of ocular tension. Accordingly, an aqueous formulation to be administered may have a temperature of less than the LCST (for example, 0° C. to less than the LCST).

More specifically, according to the disclosure of the invention, there is provided (a) a method for treating a subject, including administering an aqueous formulation containing a temperature responsive polymer to an eyeball (particularly, the anterior chamber of the eyeball) of the subject (hereinafter referred to as “method (a)”). According to the disclosure of the invention, there is provided an aqueous formulation (a formulated aqueous solution) containing a temperature responsive polymer for use in method (a). In the specification, “aqueous formulation” has a composition suitable for intraocular injection. In method (a), when an aqueous formulation containing a temperature responsive polymer is administered to an eyeball (particularly, the anterior chamber of the eyeball) of a subject, the ocular tension of the eyeball administered with the formulation can be increased.

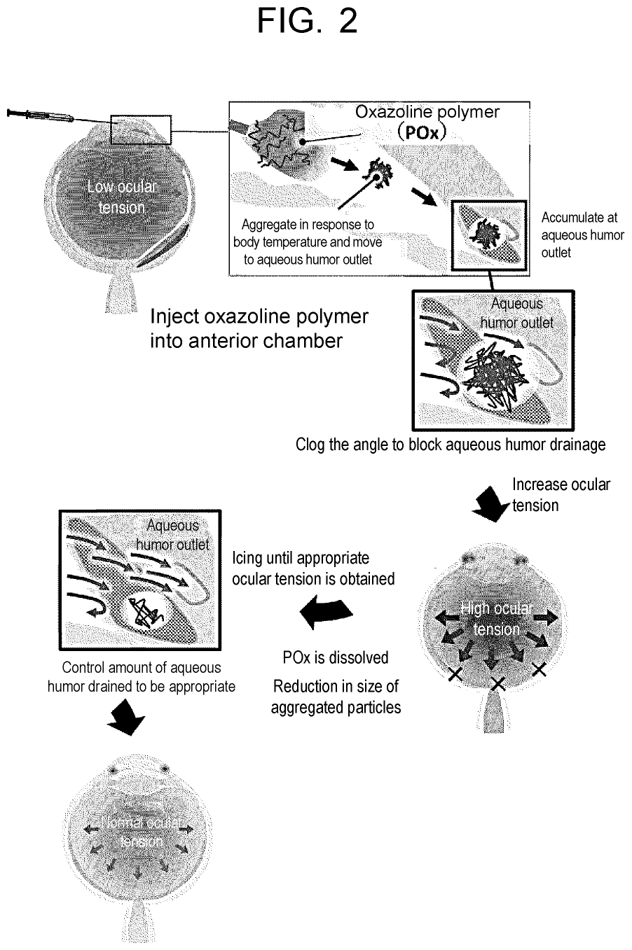

FIG. 2 schematically shows the effect of the ocular tension increasing agent of the present invention. A temperature responsive polymer has a nature of aggregating at the temperature of the intraocular circulating fluid present in the ocular tissue of a subject and being linearized as the temperature of the intraocular circulating fluid decreases. The aggregation of the temperature responsive polymer may be induced by shifting the hydrophilicity/hydrophobicity balance of the temperature responsive polymer towards hydrophobicity. The linearization of the temperature responsive polymer may be induced by shifting the hydrophilicity/hydrophobicity balance of the temperature responsive polymer towards hydrophilicity. The temperature responsive polymer may be an assembly of a plurality of temperature responsive polymer molecules or a single polymer molecule. The assembly of a plurality of temperature responsive polymer molecules may consist of different types of temperature responsive polymer molecules or same types of temperature responsive polymer molecules.

The temperature responsive polymer to be used in the method disclosed in the present invention may have the LCST within the temperature range of 0° C. to the body temperature (for example, 36° C. to 37° C.). For example, the temperature responsive polymer to be used in the method disclosed in the present invention may have the LCST of 0° C. or more, 1° C. or more, 2° C. or more, 3° C. or more, 4° C. or more, 5° C. or more, 6° C. or more, 7° C. or more, 8° C. or more, 9° C. or more, 10° C. or more, 11° C. or more, 12° C. or more, 13° C. or more, 14° C. or more, 15° C. or more, 16° C. or more, 17° C. or more, 18° C. or more, 19° C. or more, 20° C. or more, 21° C. or more, 22° C. or more, 23° C. or more, 24° C. or more, 25° C. or more, 26° C. or more, 27° C. or more, 28° C. or more, 29° C. or more, 30° C. or more, 31° C. or more, 32° C. or more, 33° C. or more, 34° C. or more, or 35° C. or more; and the body temperature or less. The temperature responsive polymer to be used in the method disclosed in the present invention may also have the LCST of, e.g., body temperature or less, 35° C. or less, 34° C. or less, 33° C. or less, 32° C. or less, 31° C. or less, 30° C. or less, 29° C. or less, 28° C. or less, 27° C. or less, 26° C. or less, 25° C. or less, 24° C. or less, 23° C. or less, 22° C. or less, 21° C. or less, 20° C. or less, 19° C. or less, 18° C. or less, 17° C. or less, 16° C. or less, 15° C. or less, 14° C. or less, 13° C. or less, 12° C. or less, 11° C. or less, 10° C. or less, 9° C. or less, 8° C. or less, 7° C. or less, 6° C. or less, 5° C. or less, or 4° C. or less. The temperature responsive polymer to be used in the method disclosed in the present invention may have the LCST of, e.g., 4° C. to 35° C., 10° C. to 30° C., 15° C. to 30° C., 20° C. to 30° C., 15° C. to 25° C., 25° C. to 30° C., 25° C. to 35° C., 20° C. to 28° C., or 20° C. to 25° C.

The temperature responsive polymer to be used in the method disclosed in the present invention may be, for example, an oxazoline polymer. The oxazoline polymer may be poly(2-oxazoline). The number average molecular weight (Mn, kDa) of the oxazoline polymer may be any value or a value between two values selected from 5, 10, 15, 20, 25, 30, 35, 40 and 45. The oxazoline polymer may be a polymer containing or consisting of a structural unit represented by the following formula (X). The oxazoline polymer may be a polymer containing or consisting of a structural unit represented by, for example, the following chemical formula (I), a copolymer (block copolymer) containing or consisting of a structural unit represented by the following chemical formula (II), a statistical copolymer containing or consisting of a structural unit represented by the following chemical formula (III) or a random copolymer containing or consisting of a structural unit represented by the following chemical formula (IV).

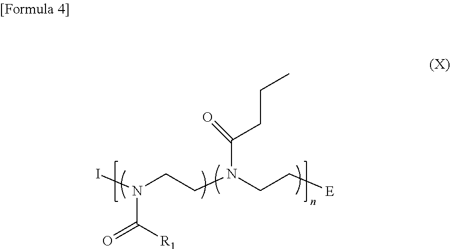

wherein R₁ represents a methyl group, an ethyl group, a propyl group (for example, a n-propyl group or an isopropyl group) or a cycloalkyl group; I represents hydrogen, alkyl, alkynyl, alkenyl, alkyloxy, a protecting group or a polymerizable group; E represents hydrogen, alkyl, alkynyl, alkenyl, alkyloxy, a protecting group or a polymerizable group; and n represents an integer of 3 to 300.

A compound represented by formula (X) may be a statistical copolymer. A compound represented by formula (X) may be a block copolymer.

In formula (X), E may be an alkyloxy, for example, a lower alkyloxy such as methoxy or ethoxy. The “lower” herein means that the number of carbon atoms is 4 or less. In formula (X), E may be a hydroxy group. In formula (X), E may be a lower alkyl group (particularly, a methyl group, an ethyl group). In formula (X), E may be CH₂═CHCH₂— or CH═CCH₂—. In formula (X), E may be H, OH—, NH₂— or N₃—.

In formula (X), I may be an alkyloxy, for example, a lower alkyloxy such as methoxy or ethoxy. The “lower” herein means that the number of carbon atoms is 4 or less. In formula (X), I may be a hydroxy group. In formula (X), I may be a lower alkyl group (particularly, a methyl group, an ethyl group). In formula (X), I may be CH₂═CHCH₂— or CH═CCH₂—. In formula (X), I may be H, OH—, NH₂— or N₃—.

In formula (X), n may be 30 to 300, for example, 50 to 150, for example, 60 to 120, for example, 70 to 110 or, for example, 80 to 100. In a preferred embodiment, n may be 50 to 150. In more preferred embodiment, n may be 60 to 120.

In formula (X), Rᵢ may be preferably a n-propyl. In formula (X), preferably, I may be hydrogen; E may be hydrogen; and n may be an 50 to 150, for example, 60 to 120, for example, 70 to 110 or, for example, 80 to 100.

In formula (X), R₁ may be preferably a n-propyl; I may be hydrogen, E may be hydrogen and n may be 50 to 150, for example, 60 to 120, for example, 70 to 110 or for example, 80 to 100.

Formula (X) may be a block copolymer. In a preferred embodiment, Formula (X) may be a statistical copolymer.

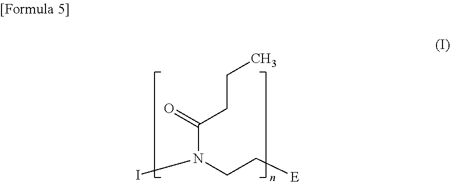

wherein n represents an integer of 30 to 300; I represents H, CH₃—, CH₂═CHCH₂— or CH≡CCH₂—; and E represents H, OH—, NH₂— or N₃—.

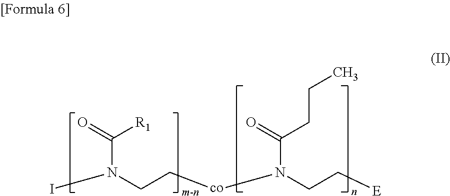

wherein R₁ represents a methyl group, an ethyl group, an isopropyl group or a cyclopropyl group; n represents an integer of 30 to 300; m and n are integers satisfying m>n and 60<n+m; I represents, H, CH₃—, CH₂═CHCH₂— or CH≡CCH₂—; and E represents H, OH—, NH₂— or N₃—.

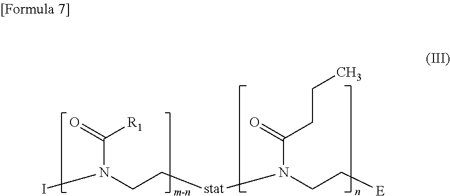

wherein
R₁ represents a methyl group, an ethyl group, an isopropyl group or a cyclopropyl group; n represents an integer of 30 to 300; m and n are integers satisfying m>n and 60<n+m; I represents H, CH₃—, CH₂═CHCH₂— or CH≡CCH₂—; and E represents H, OH—, NH₂— or N₃—.

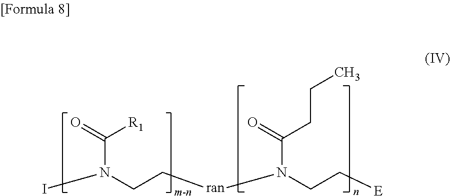

wherein
R₁ represents a methyl group, an ethyl group, an isopropyl group or a cyclopropyl group; n represents an integer of 30 to 300; m and n are integers satisfying m>n and 60<n+m≥300; I represents H, CH₃—, CH₂═CHCH₂— or CH≡CCH₂—; and E represents H, OH—, NH₂— or N₃—.

The oxazoline polymer may be a statistical copolymer or random copolymer of ethyloxazoline (also referred to as poly(2-ethyl-2-oxazoline)) and n-propyl oxazoline (also referred to as (2-propyl-2-oxazoline)).

The oxazoline polymer may be a statistical copolymer consisting of structural units represented by the following chemical formula (V).

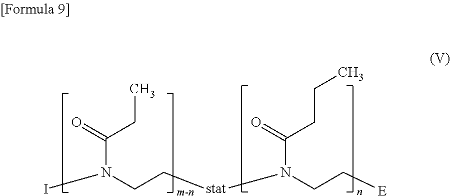

wherein
n represents an integer of 30 to 300; m and n are integers satisfying m>n and, 60<n+m; I represents H, CH₃—, CH₂═CHCH₂— or CH≡CCH₂—; and E represents H, OH—, NH₂— or N₃—.

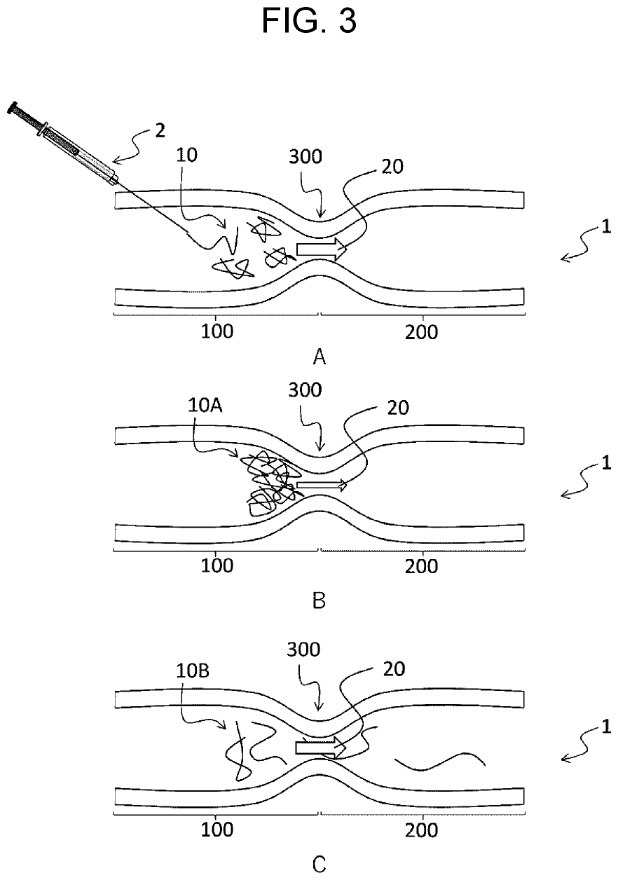

FIG. 3A is a schematic diagram of a temperature responsive polymer 10 administered within the ocular tissue 1. The ocular tissue 1 is a tissue directly or indirectly involved in or having an effect on control of ocular tension. The ocular tissue 1 of a subject is constituted of a first region 100 and a second region 200 fluidly communicated with the first region 100. The intraocular circulating fluid flows in the direction pointed by an arrow 20, more specifically, flows from the first region 100 to the second region 200. An opening portion 300 is positioned in the boundary region between the first region 100 and the second region 200. The cross section of the first region 100 is larger than that of the opening portion 300. The cross section of the second region 200 may be smaller or larger than or equal to that of the opening portion 300. The first region 100 may be the angle. The second region 200 may be Schlemm's canal. The intraocular circulating fluid is, for example, aqueous humor.

An ocular tension increasing agent containing the temperature responsive polymer 10 is administered to the first region 100, for example, by a syringe 2. The ocular tension increasing agent may be injected at a temperature at which the temperature responsive polymer 10 is aggregated or at about the same temperature as the intraocular circulating fluid. The temperature responsive polymer 10 administered to the first region 100 is aggregated at the temperature of the intraocular circulating fluid present in the ocular tissue 1 of a subject. The ocular tension increasing agent may contain the temperature responsive polymer in an amount that blocks the flow of the intraocular circulating fluid from the first region 100 to the second region 200. The phrase “block the flow of the intraocular circulating fluid” contains a nuance of “completely stopping flow of intraocular circulating fluid” and a nuance of “delaying the flow velocity of intraocular circulating fluid to the extent that ocular tension increases”.

FIG. 3B is a schematic diagram showing that a temperature responsive polymer 10A aggregated has an effect on the flow of intraocular circulating fluid in the ocular tissue 1 of a subject. The temperature responsive polymer 10A aggregated moves toward the opening portion 300 along the flow of the intraocular circulating fluid and partially or completely block the opening portion 300. Since temperature responsive polymer 1A aggregated partially or completely block the opening portion 300, the flow of the intraocular circulating fluid from the first region 100 to the second region 200 is blocked by the temperature responsive polymer aggregated. Since the flow of the intraocular circulating fluid is blocked, the ocular tension increases.

FIG. 3C shows a temperature responsive polymer 10B linearized. When the temperature of the intraocular circulating fluid in the ocular tissue 1 is decreased by a low-temperature substance such as ice and a cooling apparatus, blockage of the flow of the intraocular circulating fluid is removed as the temperature responsive polymer 10A aggregated is linearized. The temperature responsive polymer 10B linearized flows along the flow of the intraocular circulating fluid into the second region 200. Since blockage of the flow of the intraocular circulating is removed, the ocular tension retunes to original pressure.

The temperature responsive polymer can form particles at a temperature of the LCST or more. The number average particle size of the particles can be, for example, approximately 1 to 18 μm. The number average particle size of the particles can be, for example, 1 to 8 μm.

A method for administering an aqueous formulation may include, inserting a first injection needle into the anterior chamber of an eyeball, removing aqueous humor, withdrawing the first injection needle from the eyeball, inserting an injection needle of a syringe (second injection needle) containing the aqueous formulation into the hole through which the first injection needle passed such that the tip of the second injection needle arrives at the anterior chamber, followed by injecting the aqueous formulation into the anterior chamber. A hole is formed by inserting the first injection needle and the second injection needle is inserted through the hole to thereby easily administer the aqueous formulation. Aqueous humor is removed by the first injection needle to keep a space for receiving the aqueous formulation to be administered. In this way, leakage of the aqueous formulation administered from the eyeball after the administration can be prevented.

According to the disclosure of the invention, there is provided (b) a method for treating a subject, including administering an aqueous formulation containing a temperature responsive polymer to an eyeball (particularly, the anterior chamber of the eyeball) of a subject and cooling the eyeball (hereinafter referred to as “method (b)”). The phrase “cooling the eyeball” may means both or either one of cooling the eyeball of a subject by a doctor and allowing a subject to cool the eyeball by oneself.

In method (b), the eyeball is cooled by a physiologically acceptable method up to a physiologically acceptable temperature. More specifically, an eyeball is cooled neither by a method that damages the eyeball nor a method toxic to the eyeball. An eyeball is cooled such that the temperature responsive polymer is dissolved. An eyeball is cooled such that ocular tension decreases. The temperature responsive polymer is dissolved in aqueous humor when the temperature of an eyeball decreases up to less than the LCST. Accordingly, an eyeball is cooled up to a temperature of less than the LCST. An eyeball can be cooled indirectly via an eyelid or directly not via an eyelid with, for example, an aqueous solution (for example, saline solution) having a temperature less than the LCST, preferably, an aqueous solution (for example, saline solution) containing ice water.

In the cooling step of method (b), the physiologically acceptable temperature may fall within the range of 0° C. to the LCST, for example, LCST—25° C. to the LCST, for example, LCST—20° C. to the LCST, or for example, LCST—15° C. to the LCST, for example, LCST—10° C. to the LCST {however, the physiologically acceptable temperature is 0° C. or more}. In the cooling step of method (b), the physiologically acceptable temperature may fall within the range of 4° C. to the LCST, 10° C. to the LCST, 15° C. to the LCST or 20° C. to the LCST. The cooling step of method (b), since it is satisfactory that the temperature of an eyeball reduces up to less than the LCST, may include cooling an eyeball up to a temperature of less than the LCST with an aqueous solution (for example, saline solution) having a temperature of less than the LCST, preferably, an aqueous solution (for example, saline solution) containing ice water.

According to the disclosure of the invention, in the method disclosed herein, the aqueous solution may further contain not only the temperature responsive polymer (hereinafter referred to as a “first temperature responsive polymer”) but also a temperature responsive polymer (hereinafter referred to as a “second temperature responsive polymer”) modified with an uncharged hydrophilic polymer.

As the uncharged hydrophilic polymer, for example, a polyalkylene glycol such as poly (lower alkylene) glycol, particularly polyethylene glycol or polypropylene glycol, and preferably polyethylene glycol (PEG), can be used. A temperature responsive polymer may be modified with an uncharged hydrophilic polymer at the terminal oxazoline monomer of the temperature responsive polymer. The second temperature responsive polymer may contain an uncharged hydrophilic polymer and a temperature responsive polymer in a molar ratio of, for example, 1:1. More specifically, the second temperature responsive polymer can be a block copolymer of an uncharged hydrophilic polymer and a temperature responsive polymer. In the second temperature responsive polymer, an uncharged hydrophilic polymer and a temperature responsive polymer can be linked directly or indirectly via a linker.

The uncharged hydrophilic polymer may have a weight average molecular weight (Mw) of, for example, 0.5 kDa to 10 kDa, 0.5 to 10 kDa, 0.5 to 5 kDa, 0.5 to 2 kDa or 0.5 to 1.5 kDa.

The second temperature responsive polymer may be preferably PEG-polyoxazoline. The “PEG-polyoxazoline” refers to a block copolymer of PEG and polyoxazoline. The polyoxazoline used in the second temperature responsive polymer may preferably have a structure represented by formula (X).

In this embodiment, the aqueous solution contains a first temperature responsive polymer and a second temperature responsive polymer. The molar ratio of the first temperature responsive polymer and the second temperature responsive polymer may be, for example, p:100−p {wherein p may fall within the range of 90 to 99, preferably 95 to 99, more preferably 97 to 99, and further preferably about 98}.

In an aspect of the disclosure, there is provided an aqueous solution containing a first temperature responsive polymer and a second temperature responsive polymer possibly in a molar ratio, for example, 90:10 to 99:1, preferably 95:5 to 99:1, more preferably 97:1 to 99:1 and most preferably about 98:about 2. The aqueous solution may have a composition preferably suitable for intraocular administration (particularly intracameral administration). In this embodiment, the first temperature responsive polymer may be preferably polyoxazoline and the second temperature responsive polymer may be PEG-polyoxazoline.

In the method of the disclosure, an aqueous solution containing a first temperature responsive polymer and a second temperature responsive polymer may be used as the aqueous solution. In the method of the disclosure, preferably, an aqueous solution containing a first temperature responsive polymer and a second temperature responsive polymer in a molar ratio of 97:1 to 99:1 can be intraocularly administered, particularly in the anterior chamber, as the aqueous solution.

In the disclosure, there can be provided (c) a method for treating a subject, including administering an aqueous formulation containing a temperature responsive polymer to an eyeball (particularly, the anterior chamber of the eyeball) of a subject and subjecting the eyeball to at least one cycle of eyeball cooling and warming treatments (hereinafter referred to as “method (c)”). The cooling herein refers to cooling from a temperature of the LCST or more to a temperature of less than the LCST, and the warming herein refers to warming from a temperature of less than the LCST to a temperature of the LCST or more. The warming can be attained by natural warming without using a special warming means or by applying a warming treatment. The warming is made by a physiologically acceptable method up to a physiologically acceptable temperature. The warming may include warming by a doctor and warming by a subject oneself, and natural warming.

The cycle of cooling and warming an eye is carried out at least once. The cooling and warming an eye is carried out, for example, twice or more. The cooling and warming an eye is carried out, for example, three times or more. With the repeat numbers of cycles of the cooling/warming an eyeball increase, the level of an increase of the ocular tension decreases. Accordingly, the ocular tension of a subject can be controlled to be appropriate by repeating cooling/warming treatment. In other words, the cycle of cooling/warming an eyeball can be repeated until the ocular tension of the subject to be an appropriate ocular tension.

According to the disclosure of the invention, there is provided an aqueous formulation or aqueous composition containing a temperature responsive polymer for use in method (a). According to the disclosure of the invention, there is provided an aqueous formulation or aqueous composition containing a temperature responsive polymer for use in method (b). According to the disclosure of the invention, there is provided an aqueous formulation or aqueous composition containing a temperature responsive polymer for use in method (c).

In an embodiment, the aqueous solution, aqueous formulation or aqueous composition is stored at normal temperature or ambient temperature (for example, 10° C. to 27° C.). In an embodiment, the aqueous solution, aqueous formulation or aqueous composition is stored at 0° C. to 10° C. and preferably 0° C. to 5° C. In an embodiment, the aqueous solution, aqueous formulation or aqueous composition is frozen. In an embodiment, the aqueous solution, aqueous formulation or aqueous composition is lyophilized. According to the disclosure of the invention, there can be provided a combination kit containing a lyophilized product of an aqueous solution, aqueous formulation or aqueous composition and water for reconstitution. The lyophilized product can be reconstituted with water for reconstitution before use. The water for reconstitution may contain an additive(s).

A first temperature responsive polymer and a second temperature responsive polymer can be mixed in an aqueous solution having a temperature of less than the LCST. These polymers are desirably mixed in an aqueous solution having a temperature of less than the LCST. The aqueous solution kept at a temperature of less than the LCST can be used after it is sufficiently stirred preferably before increasing the temperature to the LCST or more. The aqueous solution kept at a temperature of the LCST or more can be used as it is or after it is cooled to a temperature of less than the LCST, sufficiently stirred, and thereafter, increased to a temperature of the LCST or more before use.

According to the disclosure of the invention, there is provided an aqueous formulation or aqueous composition containing a first temperature responsive polymer and a second temperature responsive polymer, for use in method (a). According to the disclosure of the invention, there is provided an aqueous formulation or aqueous composition containing a first temperature responsive polymer and a second temperature responsive polymer, for use in method (b). According to the disclosure of the invention, there is provided an aqueous formulation or aqueous composition containing a first temperature responsive polymer and a second temperature responsive polymer, for use in method (c). The first temperature responsive polymer and second temperature responsive polymer herein are the same as defined in the above.

According to the disclosure of the invention, there is provided use of a temperature responsive polymer in manufacture of a composition or a formulation, for use in method (a). According to the disclosure of the invention, there is provided use of a temperature responsive polymer in manufacture of a composition or a formulation for use in method (b). According to the disclosure of the invention, there is provided use of a temperature responsive polymer in manufacture of a composition or a formulation for use in method (c).

According to the disclosure of the invention, there is provided use of a first temperature responsive polymer and a second temperature responsive polymer in producing a composition or a formulation, for use in method (a). According to the disclosure of the invention, there is provided use of a first temperature responsive polymer and a second temperature responsive polymer in producing a composition or a formulation, for use in method (b). According to the disclosure of the invention, there is provided use of a first temperature responsive polymer and a second temperature responsive polymer in producing a composition or a formulation, for use in method (c).

In the method disclosed herein, the subject may be preferably a subject having a low ocular tension, for example, a subject having ocular hypotension. The subject having low ocular tension or ocular hypotension may or may not have a retinal disorder such as detachment of ciliary body, retinal detachment and macular degeneration, or iridocyclitis. The subject having low ocular tension or ocular hypotension may or may not have a complication.

Composition or Pharmaceutical Composition for Use in Treating Ocular Hypotension

As described above, according to the method disclosed herein, it is possible to increase the ocular tension of a subject. Thus, according to the method disclosed herein, it is possible to treat ocular hypotension in a subject in need thereof. In this embodiment, a subject can be treated by method (a), (b) or (c). According to the method disclosed herein, it is possible to reduce an onset risk of a complication or prevent a complication by the method.

A composition or a pharmaceutical composition according to the embodiment for use in treating ocular hypotension contains an ocular tension increasing agent as mentioned above. The composition or a pharmaceutical composition according to the embodiment for use in treating ocular hypotension contains, for example, a temperature responsive polymer or a first temperature responsive polymer and a second temperature responsive polymer. The composition or pharmaceutical composition of the present invention is administered by injection to the first region 100.

The composition or pharmaceutical composition of the present invention may further contain a pharmacologically or pharmaceutically acceptable additive. The optimal concentrations of a pharmacologically or pharmaceutically acceptable additive and an additive to be used in the present invention may be experimentally easily selected/determined by those skilled in the art. Examples of the pharmaceutically acceptable additive for use in the present invention include a solvent, a buffer, a chelating agent, an antioxidant and/or a viscosity enhancer.

The pharmacologically or pharmaceutically acceptable additive refers to a substance substantially nontoxic to an individual to which the composition or pharmaceutical composition is to be administered. Such an additive usually satisfies the requirements defined by government institution responsible for drugs. Regulations on pharmaceutically acceptable additives known in the art are specified in official pharmacopoeia such as US Pharmacopeia and EP Pharmacopoeia.

Examples of the solvent include, but are not limited to, water and alcohol. Water is preferable. Examples of the buffer include, but are not limited to, citric acid, acetic acid, tartaric acid, lactic acid, bicarbonate, phosphate and diethylamine. Examples of the chelating agent include, but are not limited to, EDTA sodium and citric acid. Examples of the antioxidant include, but are not limited to, butylated hydroxyl anisole (BHA), ascorbic acid and a derivative thereof, tocopherol and a derivative thereof, cysteine and a mixture of these. Examples of the viscosity enhancer include, but are not limited to, polyvinyl alcohol, polyvinylpyrrolidone, methyl cellulose, hydroxypropyl methylcellulose, hydroxyethyl cellulose, carboxymethyl cellulose and hydroxypropyl cellulose.

The composition or pharmaceutical composition of the present invention has pH within the range of 4.5 to 8.0 (particularly, within the range of 5.0 to 6.0, 6.0 to 7.0 or 7.0 to 8.0, preferably within the range of pH 7.4 to 7.8, preferably pH 7.6). The composition or pharmaceutical composition of the present invention is preferably prepared so as to have an osmotic pressure value equivalent to that of aqueous humor of an eye and an ocular tissue. The osmotic pressure value ordinarily falls within the range of about 200 to about 400 milliosmol/kilogram (“mOsm/kg”), preferably about 250 to about 350 mOsm/kg, and preferably about 300 mOsm/kg.

The composition or pharmaceutical composition of the present invention can be provided in the form of a prefilled syringe. The prefilled syringe may include a prefilled syringe containing the composition or pharmaceutical composition of the present invention. The prefilled syringe may be aseptically packaged. The prefilled syringe may have a syringe cap or a needle. In this embodiment, the composition or pharmaceutical composition of the present invention is prepared for an injection.

Method for Treating Ocular Hypotension

A method for treating ocular hypotension according to the embodiment has a step of administering an ocular tension increasing agent as mentioned above, or the composition or pharmaceutical composition of the present invention to an ocular tissue of a subject requiring a treatment of ocular hypotension. The ocular tension increasing agent as mentioned above, or the composition or pharmaceutical composition of the present invention can be administered by injection to the ocular tissue 1. The ocular tension increasing agent as mentioned above, or the composition or pharmaceutical composition of the present invention is administered to the first region 100 of the ocular tissue 1.

In the embodiment, there is provided an ocular tension increasing agent containing a temperature responsive polymer. However, the agent can be applied to a tissue (for example, capillaries) other than an ocular tissue.

According to the disclosure of the invention, there can be provided use of a temperature responsive polymer in manufacture of a composition or pharmaceutical composition for use in a method for treating ocular hypotension in a subject having ocular hypotension. According to the disclosure of the invention, there can be provided use of a first temperature responsive polymer and a second temperature responsive polymer in manufacture of a composition or pharmaceutical composition for use in a method for treating ocular hypotension in a subject having ocular hypotension. According to the disclosure of the invention, there can be provided use of an aqueous formulation mentioned above containing a first temperature responsive polymer and a second temperature responsive polymer in manufacture of a composition or pharmaceutical composition for use in a method for treating ocular hypotension in a subject having ocular hypotension. A method for treating ocular hypotension in a subject having ocular hypotension may include method (a) {however, a subject has ocular hypotension}. A method for treating ocular hypotension in a subject having ocular hypotension may include method (b) {however, a subject has ocular hypotension}. A method for treating ocular hypotension in a subject having ocular hypotension may include method (c) {however, a subject has ocular hypotension}.

According to the disclosure of the invention, there can be provided a temperature responsive polymer for use in a method for treating ocular hypotension in a subject having ocular hypotension. According to the disclosure of the invention, there can be provided a first temperature responsive polymer and a second temperature responsive polymer for use in a method for treating ocular hypotension in a subject having ocular hypotension. According to the disclosure of the invention, there can be provided an aqueous formulation containing a first temperature responsive polymer and a second temperature responsive polymer for use in a method for treating ocular hypotension in a subject having ocular hypotension.

### EXAMPLES

### Example 1: Observation of an Increase in Ocular Tension by Administration of Composition Containing an Oxazoline Polymer (Hereinafter Sometimes Referred to as “POx”) and a Decrease in Ocular Tension by Cooling Eyeball after Administration

### Experimental Method

An oxazoline polymer (Ultroxa®, poly(2-propyl-2-oxazoline), serial number: 900352-1G, Sigma-Aldrich; average Mn: 10,000; n=89) and a saline solution were mixed on ice to prepare a 10% (wt/wt) aqueous solution (aqueous oxazoline solution). To the right-eye anterior chamber of each of closed colony (SD) rats anesthetized with ketamine and xylazine, the aqueous oxazoline solution (5 μL) was injected by use of a micro syringe. After treatment, infection and dry eye were prevented with Tarivid ophthalmic ointment.

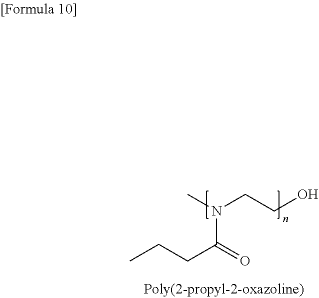

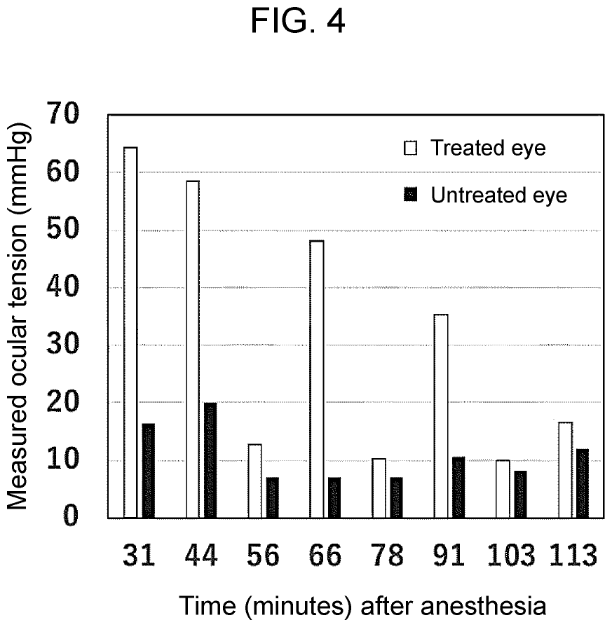

The ocular tensions of the treated eye (right eye) and the untreated eye (left eye) were measured with time under anesthesia 10 days after injection of the aqueous oxazoline solution (FIG. 4). Since tonometry data significantly fluctuate, a measurement was repeated 5 times. The ocular tension of the untreated eye was 7 to 17 mmHg during a measurement period. However, in the eye treated by injection with an aqueous oxazoline solution, a significant increase (55 mmHg or more) of ocular tension was observed 31 minutes and 44 minutes after anesthesia. After the eye was cooled for 10 minutes thereafter, and ocular tension was measured 56 minutes after anesthesia. As a result, the ocular tension of the treated eye reduced to 12 mmHg. When the treated eye was allowed to stand still at normal temperature for 10 minutes, ocular tension increased again to 48 mmHg (66 minutes after anesthesia). Thereafter, a cycle of cooling/stand-still at room temperature was repeated twice. As a result, ocular tension significantly decreased after cooling similarly to the above and returned to a high value after standing still at room temperature. It was found that the ocular tension tends to be lower on and after the second time than the first time. From the above, it was demonstrated that an initial purpose: increasing ocular tension by administration of the aqueous oxazoline solution and decreasing the ocular tension by cooling, can be attained. An increase and decrease of ocular tension can be repeatedly induced; that is, an increase and decrease of ocular tension was found to be reproducible. In addition, it was apparent that the ocular tension increased after it was once decreased is lower than that of the previous time. One of the reasons of this is considered because the polymer was dissolved in aqueous humor by cooling and flew out, with the result that the amount of the polymer in the anterior chamber decreased.

In contrast, since there was a possibility that the result of an ocular-tension increase induced by polyoxazoline may vary depending on technique/ability of the operator, we decided to improve the composition of a formulation.

### Example 2: Synthesis of Copolymer of Uncharged Hydrophilic Polymer and Oxazoline Polymer

A copolymer of an uncharged hydrophilic polymer and an oxazoline polymer was synthesized as follows. Solubility of the oxazoline polymer in water is considered to increase by copolymerizing it with an uncharged hydrophilic polymer.

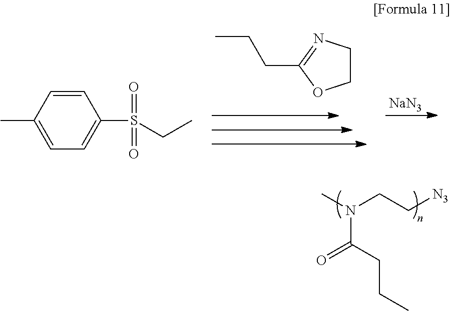

Acetonitrile containing n-propyl oxazoline (nPrOx) was dewatered with calcium hydride and purified by vacuum distillation or atmospheric distillation. p-Methyl toluenesulfonate (TsOMe) was dewatered with diphosphorus pentaoxide and purified by vacuum distillation. TsOMe (50 μL, 0.33 mmol) and nPrOx (3.9 mL, 33.1 mmol) were dissolved in acetonitrile (10 mL) and the mixture was stirred at 42° C. for 7 days. Sodium azide (215 mg, 3.3 mmol) was added as a polymerization terminator and the mixture was stirred at 70° C. for 24 hours. These operations were all carried out in an argon atmosphere. The polymerization solution obtained was dialyzed against methanol and water separately three times (MWCO=3500) and lyophilized to obtain PnPrOx-N₃ (molecular weight: 8000).

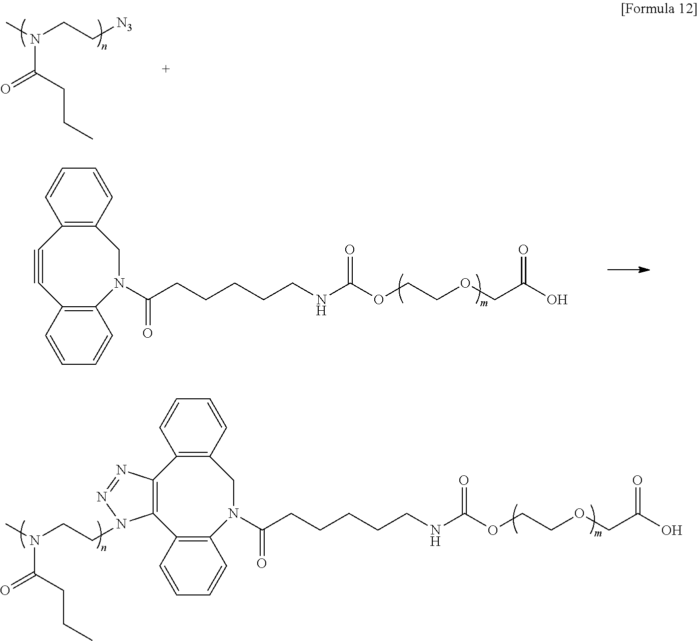

PnPrOx-N₃ (400 mg) having a molecular weight of 8000 and DBCO-PEG-COOH (100 mg) having MW of 1000 were dissolved in methanol (6 mL). The mixture was stirred at 40° C. for two days and dialyzed against methanol and water separately three times (MWCO=3500) and lyophilized to obtain a desired product (hereinafter referred to as “POxPEG”). The LCSTs of POx and POxPEG fell within the range of 20° C. to 25° C.

### Example 3: Preparation of a Mixture of Pox and POxPEG and Evaluation of the Physical Properties of the Mixture

Aqueous solutions containing POx and POxPEG in the indicated ratios (weight ratio) were obtained and observed at 37° C. More specifically, a saline solution containing 1 wt % of POx and a saline solution containing 1 wt % of POxPEG were prepared on ice. Both saline solutions were mixed well in various ratios at 4° C. to obtain POx/POxPEG mixed solutions. Onto a first slide glass, a 100 μm-thick silicon rubber sheet having a hole in the center was allowed to adhere. Each of the POx/POxPEG mixed solutions was added to the hole of the center and covered with a second slide glass. On the second slide glass, a transparent heater was placed. The POx/POxPEG mixed solutions were observed from the bottom by an inverted microscope.

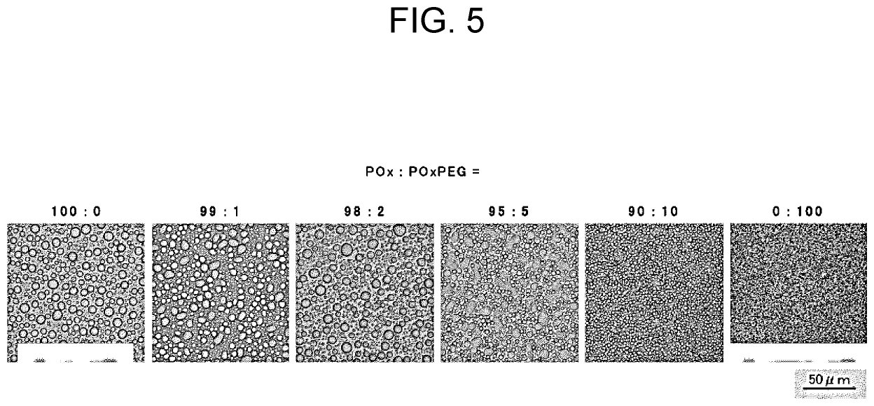

First, the temperature of the heater was set at 37° C. and microscope images were obtained. The results are as shown in FIG. 5. As is shown in FIG. 5, in the saline solution containing POx alone, POx particles having a relatively large particle size adhered on the slide glass. In contrast, in the saline solution containing POxPEG alone, particles having a relatively small particle size floated without adhering to the slide glass. The intermediate characteristics between them were observed in accordance with the mixing ratios of the particles.

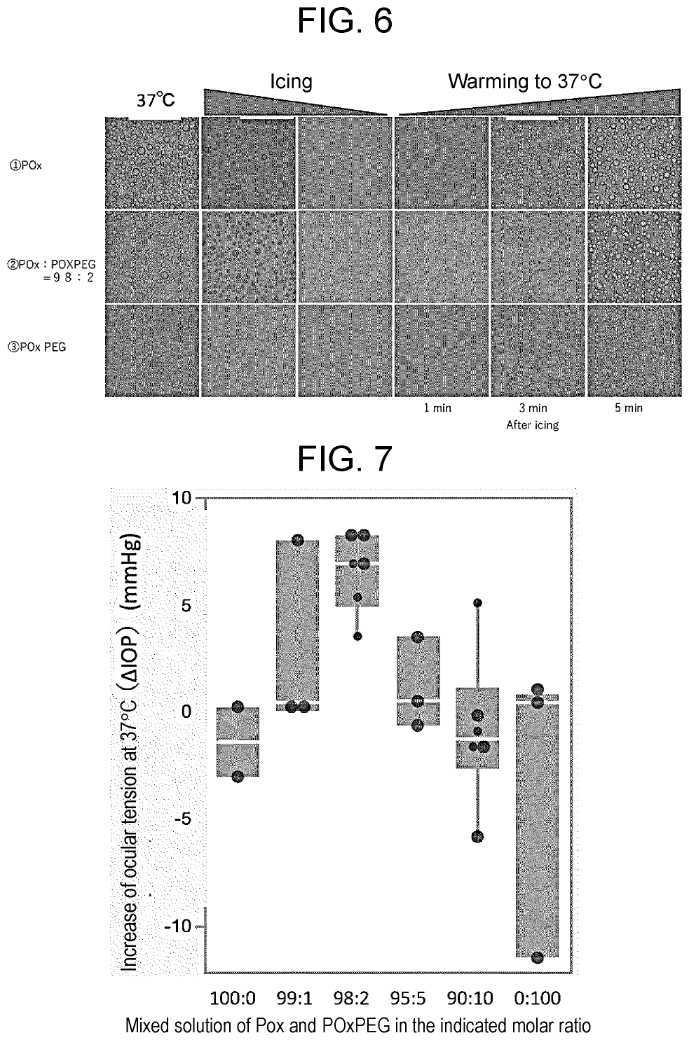

Next, experiments by cooling were carried out. The results are as shown in FIG. 6. As shown in FIG. 6, the heater on the slide glass was kept “on”, a bag containing ice water was placed on the upper part to cool (icing) a sample containing portion. As a result, it was confirmed that particles observed were dissolved and disappeared. From this, it was suggested that the particles obtained can be removed by cooling. When the bag containing ice water was removed and the temperature of the sample containing portion was returned to 37° C., particles having the same shape as that before cooling were reconstituted. From these results, it was suggested that drainage of aqueous humor from the aqueous humor outlet can be blocked by administration of the mixed solution obtained to an eyeball. From these results, it was also suggested that blockage of aqueous humor drainage can be cancelled by cooling, and that ocular tension can be decreased again in this manner.

### Example 4: Experiment of Increasing and Decreasing Ocular Tension

White house rabbits (2 to 3.5 kg) were positioned with a positioner and ocular tension was measured by a handy tonometer (Icare TONOVET Plus, M.E, Technica) while they were kept awake, and then anesthetized with ketamine and xylazine (ketamine hydrochloride 60 mg/kg; Sankyo, Tokyo, Japan and xylazine 10 mg/kg; Bayer, Munich, Germany). Ocular tension was again measured, and eyeballs were observed by a microscope under anesthesia. Thereafter, 100 μL of 1 wt % POxMix was injected into the anterior chamber of just one of the eyes of each rabbit. Microscopic observation and tonometry were carried out immediately after injection, 30 minutes and 24 hours after anesthesia.

The results are as shown in FIG. 7. As shown in FIG. 7, it was confirmed that there were house rabbits whose ocular tension increased at a ratio of POx:POxPEG of 0:100 to 99:1 or 90:10 to 99:1 but the level of increase was low in some of the house rabbits. In the case where POx:POxPEG is 98:2, it was confirmed that an increase in ocular tension is particularly excellent. In this mixing ratio, a variation in the level of increase in ocular tension depending on the operator's technique was not virtually observed and administration was satisfactory.

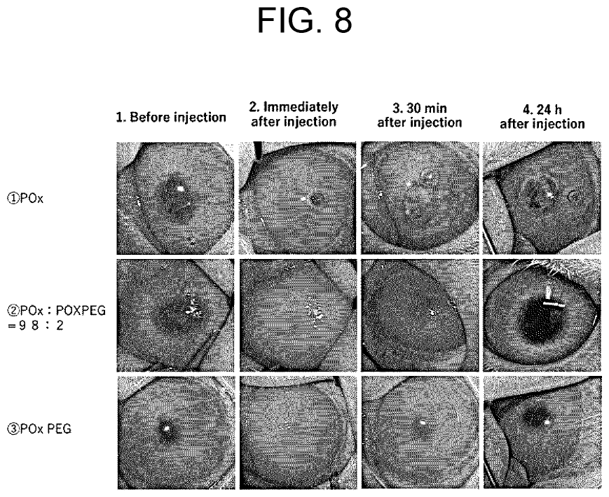

When a saline solution containing an oxazoline polymer used in Example 1 was administered, the eyeball became cloudy immediately after administration, and the oxazoline polymer deposited on the iris and lens surface with the passage of time and remained as a white sediment even after 24 hours (see, FIG. 8, upper panel). When a saline solution containing POxPEG was administered, eyeballs also became cloudy immediately after administration but the degree of cloudiness decreased 30 minutes later, and ocular tension did not increase (FIG. 7), although the eyeballs became transparent after 24 hours (see, FIG. 8, lower panel). In contrast, in the case of eyeballs administered with a saline solution containing POx:POxPEG (98:2), even though the eyeballs turned cloudy immediately after administration similarly to the case of POxPEG and the degree of cloudiness decreased 30 minutes later. After 24 hours, not only did cloudiness virtually disappear but also ocular tension increased as well (FIG. 7). The particles of a temperature responsive polymer cause cloudiness but move to the angle in the case of POx:POxPEG (98:2), with the result that particles on lens and the iris disappeared. This was considered as a reason why the degree of cloudiness decreased. The reason why ocular tension did not increase in the case of POxPEG and increased in the case of POx:POxPEG (98:2) is considered as follow: In the former case, since the particle size is small, the particles did not remain at the angle and were drained. In the case of POx:POxPEG (98:2), since the particle size is large, the particles remained at the angle.

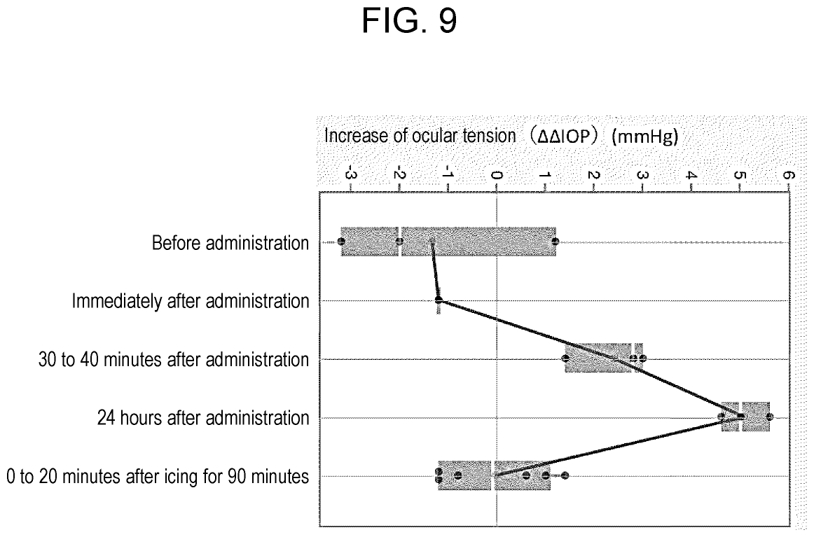

When ocular tension of house rabbits administered with a saline solution containing 1 wt % POx:POxPEG (98:2) was measured, ocular tension increased by approximately 2.5 mmHg 30 to 40 minutes after injection and approximately 5 mmHg 24 hours later, as shown in FIG. 9. From these results, it was suggested that an ocular tension increase effect was achieved in eyeballs from which cloudiness disappeared.

Thereafter, the house rabbits were positioned by a positioner and the eyeballs were cooled with ice water for 90 minutes, and then, ocular tension was measured. As a result, ΔΔIOP decreased to 0 mmHg. From the result, it was demonstrated that ocular tension can be decreased by cooling.

1. A method for modulating ocular tension in a subject, comprising administering a temperature responsive polymer to an anterior chamber of an eyeball in the subject to increase the ocular tension of the subject, and cooling the eyeball that has received the temperature responsive polymer to decrease the ocular tension.

2. The method according to claim 1, wherein the temperature responsive polymer has a lower critical solution temperature (LCST) between 0° C. and 37° C.

3. The method according to claim 2, wherein the temperature responsive polymer has a lower critical solution temperature (LCST) between 15° C. and 30° C.

4. (canceled)

5. The method according to claim 1, further comprising administering a copolymer of an uncharged hydrophilic polymer and a temperature responsive polymer.

6. The method according to claim 1, wherein the temperature responsive polymer is polyoxazoline.

7. The method according to claim 5, wherein the uncharged hydrophilic polymer is a polyalkylene glycol.

8. A composition comprising polyoxazoline (POx) and a block copolymer (POxPEG) comprising a polyethylene glycol block and a polyoxazoline block.

9. The composition according to claim 8, wherein a weight ratio of POx to POxPEG is from 90:10 to 99:1.

10. A method for increasing ocular tension in a subject, comprising administering a temperature responsive polymer of claim 8 to an eyeball of a subject.

11. A method for increasing ocular tension in a subject, comprising administering a temperature responsive polymer of claim 9 to an eyeball of a subject.

12. The method according to claim 11, wherein the temperature responsive polymer has a lower critical solution temperature (LCST) between 0° C. and 37° C.

13-14. (canceled)

15. The method according to claim 10, wherein the temperature responsive polymer has a lower critical solution temperature (LCST) between 0° C. and 37° C.

16. The method according to claim 2, further comprising administering a copolymer of an uncharged hydrophilic polymer and a temperature responsive polymer.

17. The method according to claim 3, further comprising administering a copolymer of an uncharged hydrophilic polymer and a temperature responsive polymer.

18. The method according to claim 2, wherein the temperature responsive polymer is polyoxazoline.

19. The method according to claim 3, wherein the temperature responsive polymer is polyoxazoline.

20. The method according to claim 5, wherein the temperature responsive polymer is polyoxazoline.

21. The method according to claim 16, wherein the temperature responsive polymer is polyoxazoline.

22. The method according to claim 17, wherein the temperature responsive polymer is polyoxazoline.

23. The method according to claim 6, wherein the uncharged hydrophilic polymer is a polyalkylene glycol.

24. The method according to claim 16, wherein the uncharged hydrophilic polymer is a polyalkylene glycol.

25. The method according to claim 17, wherein the uncharged hydrophilic polymer is a polyalkylene glycol.

26. The method according to claim 20, wherein the uncharged hydrophilic polymer is a polyalkylene glycol.

27. The method according to claim 21, wherein the uncharged hydrophilic polymer is a polyalkylene glycol.

28. The method according to claim 22, wherein the uncharged hydrophilic polymer is a polyalkylene glycol.
